(19) United States
(12) Patent Application Publication
(10) Pub. No.: US 2009/0011984 A1
(43) Pub. Date: Jan. 8, 2009

(54) Biotin-binding receptor molecules

(76) Inventors: Seppo Yla-Herttuala, Kuopio (FI); Markku Kulomaa, Tampere (FI); Pauliina Lehtolainen, Kuopio (FI); Varpu Marjomaki, Jyvaskyla (FI); Kari Airenne, Kuopio (FI)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/789,739

(22) Filed: Apr. 24, 2007

Related U.S. Application Data
(63) Continuation-in-part of application No. 10/618,570, filed on Jul. 11, 2003, now Pat. No. 7,208,291.
Application No. 10/618,570 is a continuation of application No. 09/622,804, filed on Aug. 22, 2000, now abandoned.

(30) Foreign Application Priority Data
Feb. 23, 1998 (GB) 9803757.5
Jun. 24, 1998 (GB) 9813653.4

(51) Int. Cl. A61K 38/00; C12N 5/06; A61P 43/00

(52) U.S. Cl. 514/12; 435/375

(57) ABSTRACT

The subject invention pertains to a transmembrane protein capable of binding to biotinylated molecules, the protein comprising a cytoplasmic domain, a membrane-spanning domain and an extracellular domain, wherein the extracellular domain comprises biotin-binding activity, and methods of use. The protein can be expressed in a cell, thereby targeting a biotinylated drug.

### CROSS-REFERENCE TO RELATED APPLICATIONS

This application is a continuation-in-part of U.S. application Ser. No. 10/618,570, filed Nov. 7, 2003, now U.S. Pat. No. 7,208,291, issued Apr. 24, 2007, which is a continuation of U.S. application Ser. No. 09/622,804, filed Aug. 22, 2000, which is the U.S. national stage of International Application No. PCT/GB99/00546, filed Feb. 23, 1999, each of which are hereby incorporated by reference in its entirety, including all figures, nucleic acid sequences, amino acid sequences, and tables.

### FIELD OF THE INVENTION

This invention relates to membrane-spanning proteins having biotin-binding activity and to their use.

### BACKGROUND TO THE INVENTION

Biotin (vitamin H) is a readily water-soluble substance found at low concentrations in blood and tissues. The biological role of biotin is as a carrier of activated CO₂, and it permits the transfer of CO₂ to acceptors without the need for additional free energy. The activated carboxybiotin is usually attached to an enzyme that is required for the formation of carboxybiotin. For example, biotin may be attached to pyruvate carboxylase which, in the presence of acetyl CoA, catalyzes the formation of carboxybiotin and the subsequent transfer of the activated carboxyl group to pyruvate, to form oxaloacetate.

Biotin also binds with one of the highest naturally known affinities to avidin, a 63 kDa glycoprotein from chicken egg white, and to streptavidin, a non-glycosylated protein from the bacterium Streptomyces avidinii. The binding is almost irreversible in character (Ka 10¹⁵ mol⁻¹). The affinity between avidin and biotin has proved very useful in a wide variety of bioanalytical applications. For example, the avidin-biotin complex has been used successfully in a wide variety of detection systems where target molecules are combined with biotin through its carboxy terminus, to form biotinylated molecules which may be easily detected or separated from solution. Biotinylation can occur without changing the biological or physiochemical properties of the various molecules and without affecting the binding capacity of the biotin prosthetic group to avidin.

WO87/05026 discloses the isolation of a DNA sequence encoding streptavidin and a fusion of the streptavidin gene and a gene encoding the human LDL receptor. The fused gene expresses a protein which consists of streptavidin at the N-terminal region of the fused protein and the LDL receptor protein at the C-terminal region of the fused protein. The fused gene may be inserted in an expression vector and used to transform a host cell. The presence of the fusion streptavidin-LDL receptor protein at a cell surface may be determined by addition of blood cells coupled to biotinylated bovine serum albumin.

Kulomaa et al., FASEB J. 9(6):A1395 (1995), discloses the construction of several avidin fusion protein vectors, including a fusion protein consisting of the avidin protein fused to the chicken progesterone receptor B which has been expressed in Escherichia coli.

Marjomaki et al., “Molecular Biology of the Cell” (December 1996), Vol. 7, supp. 5, pp 2631, pub. Am. Chem. Soc. Cell Bio., discloses the use of a Semliki forest virus expression system in order to obtain the transient expression of a chimeric protein containing the transmembrane domain and part of the cytoplasmic domain of the cation-independent mannose 6-phosphate receptor fused to a recombinant avidin in BHK cells.

### BRIEF SUMMARY OF THE INVENTION

It has now been realized that the biotin-binding activity of avidin and streptavidin may be utilized in the production of transmembrane proteins capable of binding biotinylated molecules.

According to a first aspect of the present invention, a fusion protein comprises a membrane-spanning domain of an endocytotic receptor and an extracellular domain that comprises biotin-binding activity.

According to a second aspect of the invention, a nucleic acid encodes a fusion protein as defined above.

Proteins of the present invention may comprise a cytoplasmic domain, a membrane-spanning domain and an extracellular domain, wherein the extracellular domain comprises biotin-binding activity. The extracellular domain may comprise avidin or streptavidin functional activity.

Using proteins or nucleic acid molecules of this invention, it is possible to target biotinylated molecules to specific sites in tissues. Molecules targeted in this way may be taken up by the tissues or cells by endocytosis, allowing the molecules to exert their effects within or on the cell.

### BRIEF DESCRIPTION OF THE DRAWINGS

FIG. 1 is a schematic illustration of a fusion protein of the present invention, where A represents avidin and B represents the membrane-spanning domain of an endocytotic receptor (and C represents biotin);

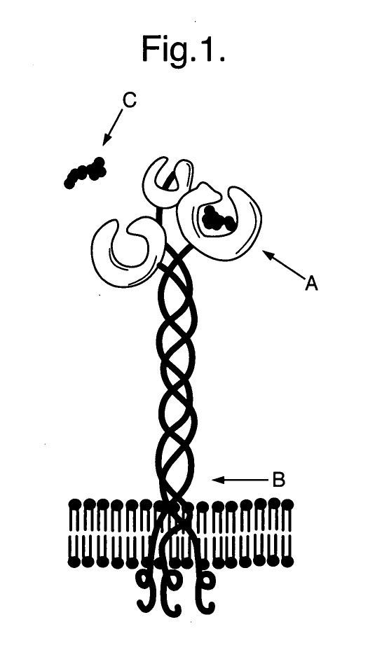

FIG. 2 is a schematic illustration of a cloning strategy using a shuttle vector; and

FIG. 3 is a schematic illustration of a cloning strategy using a retrovirus vector.

### BRIEF DESCRIPTION OF THE SEQUENCES

SEQ ID NO: 1 is of a polynucleotide encoding the protein of SEQ ID NO: 2.

SEQ ID NO: 2 is of a fusion protein of the scavenger receptor and avidin.

SEQ ID NO: 3 is of a polynucleotide encoding a functional fusion protein of the LDL receptor and avidin.

SEQ ID NO: 4 is of a fusion protein of the invention.

SEQ ID NO: 5 is of a fusion protein of the invention.

### DETAILED DISCLOSURE OF THE INVENTION

Proteins and polynucleotides for use in the present invention may be produced using conventional recombinant DNA technology. Typically, a DNA sequence coding for the functional domain of a biotin-binding protein such as avidin, streptavidin or a related protein, is engineered into a genetic construct which comprises a DNA sequence coding for a protein having membrane-spanning properties. Examples of avidin and streptavidin-related proteins include AVR-1-AVR-5, AVR-X-AVR-V, Stv1 and Stv2.

The individual domains of the fusion protein may be amplified by polymerase chain reaction or isolated from the parent cDNA using restriction enzyme digestion, isolation and purification, e.g., using gel electrophoresis, and subsequent ligation, e.g., using DNA ligase. The fusion protein construct may then be transfected into any suitable host cell, cultured and isolated using standard protein purification techniques.

The construct may also be used as naked DNA or as a plasmid/liposome, plasmid/polyethyleneimine, plasmid/dendrimer or plasmid/peptide complex.

Alternatively, the construct may be introduced into a replication-deficient virus which can be used to target the construct to specific sites in vivo. For example, the construct may be a retroviral vector comprising the appropriate cDNA for the fusion protein. A replication-deficient retrovirus, e.g., Moloney murine retrovirus, may then be used for the stable transfection of target cells and tissues. Other viruses that can be used include replication-deficient adenoviruses, adeno-associated viruses, herpes viruses, papilloma viruses and sinibis viruses. Additional viruses will be apparent to those skilled in the art.

In addition to the functional domains of avidin, streptavidin or related protein, the fusion protein will typically comprise the membrane-spanning domains of endocytotic receptors. The use of these receptors enables the uptake of biotinylated molecules into a target cell. Suitable receptors that may be used in this invention include the scavenger receptor class A, low density lipoprotein (LDL) receptor, very low density lipoprotein receptor, transferrin receptor and the LOX-1 receptor. The fusion protein may also comprise a linker between the receptor protein and the avidin peptide sequences. The linker may be any length, provided that the functional activity of the different components of the fusion protein is retained.

In general, the fusion between avidin or streptavidin peptide sequences and the receptor peptide sequences is between the extracellular domain of the receptor protein and any site outside of the biotin-binding site of avidin or streptavidin.

Any of a variety of drugs may be biotinylated, for use in the invention. Procedures for biotinylation are known to those of ordinary skill in the art. The drug itself may be chemically modified by biotin, or biotin may be attached, e.g., chemically, to a carrier molecule or particle. The drug may be a cytotoxic agent such as methotrexate or 5-fluorouracil, or a radioactive compound or atom such as Tc or ⁹⁰Y. For example, DTPA molecules or DOTA can be used as a ligand (“carrier”) for yttrium. The advantage of DTPA molecules is that they have two biotin moieties (instead of one) and they can be engineered to form bigger complexes. Potentially this could result in an increase in therapeutic efficacy.

The drug will be chosen having regard to the condition to be treated. The condition is, for example, cancer. The subject to be treated may be hosting a tumor.

The route of administration, the formulation of the drug and its dosage can each be chosen by one of ordinary skill in the art, having regard to the usual factors such as the potency of the drug, the nature and severity of the condition, the condition of the patient etc. For example, an optimal treatment schedule might comprise Yttrium given once a week, twice a week or every second week. The dosage can be based on understanding the amount of total activity to be given. For example, in humans a total amount of 60 Gray is normally given to patients with malignant glioma. This amount is divided into many small dosages that are given following a specific schedule (treatment schedule) to the patient.

An interesting potential application of the invention is in combination with the gene therapy described in U.S. Pat. No. 6,579,855. That gene therapy is based on the introduction of the Herpes Simplex Virus thymidine kinase (HSV-tk) to the region of a tumor. Cells transduced with the gene for thymidine kinase produce the protein HSV-tk. Thymidine kinase transforms ganciclovir, which as such is non-toxic, into a metabolite that kills tumor cells. Combination with the gene therapy described herein may provide an additive/synergistic therapeutic effect. This can be implemented by two approaches. The first approach is to use two different gene transfer vectors for gene transfer. This requires that each of the gene therapy has to be applied separately. The second is to clone both gene therapies into a single vector. This can be done either by having both genes within a single expression cassette or by having both genes separately within an independent expression cassette.

As gene transfer vectors, adenovirus, lentivirus, baculovirus, or adeno-associated viruses can be used. Non-viral vectors can also be used.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

A DNA construct was created between the bovine scavenger receptor class A (ScR) (Kodama et al. (1990) Nature 343:531-535) and avidin (Green (1975) Adv. Prot. Chem. 29:85-133), which codes for a protein having a ScR cytoplasmic domain, membrane-spanning domain and α-helical coiled domain, ligated to a biotin-binding domain. The complete amino acid sequence of the fusion protein is shown in SEQ ID NO: 2 where amino acids 1-53 represent the cytoplasmic domain; amino acids 55-79 represent the transmembrane domain; amino acids 81-111 represent a spacer domain; and amino acids 113-272 represent the α-helical coiled domain. Amino acids 273-400 represent the mature avidin peptide sequence derived from avidin cDNA (Gope et al. (1987) Nucleic Acid Res. 15:3595-3606) lacking a secretion signal.

Briefly, the cDNA for ScR was obtained from cultured cells previously transfected with a plasmid (PLScRNL) containing the ScR cDNA with an internal Rous Sarcoma Virus promoter and HindIII restriction sites. The isolated cDNA was then inserted into a HindIII site of the retrovirus vector pLS1ARNL. The avidin cDNA was produced by the polymerase chain reaction and then inserted into the retrovirus vector at a Sty1 restriction site on the ScR cDNA. The cDNA embodying the invention is shown as SEQ ID NO: 1, where nucleotides 1-989 represent a long terminal repeat from Mo-MuSV; nucleotides 1071-2270 represent the coding region for the fusion protein; nucleotides 2376-3101 represent an untranslated region from bovine scavenger receptor I cDNA; nucleotides 3107-3376 represent an RSV promoter region; nucleotides 3727-4522 represent aneo R gene; and nucleotides 4540-5177 represent a long terminal repeat from Mo-MuLV.

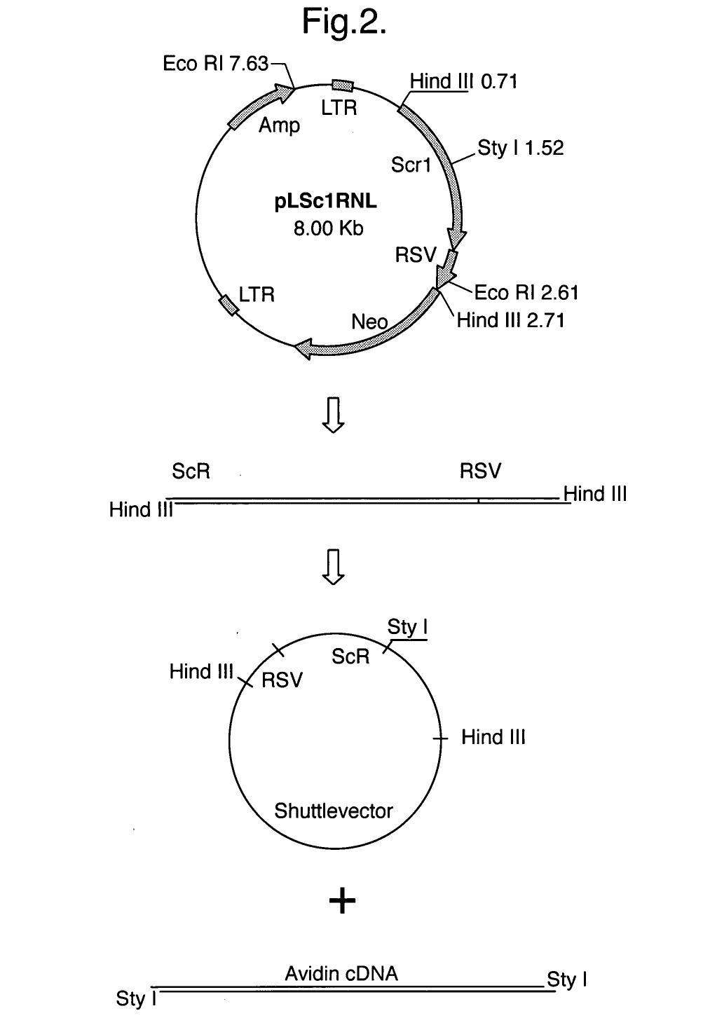

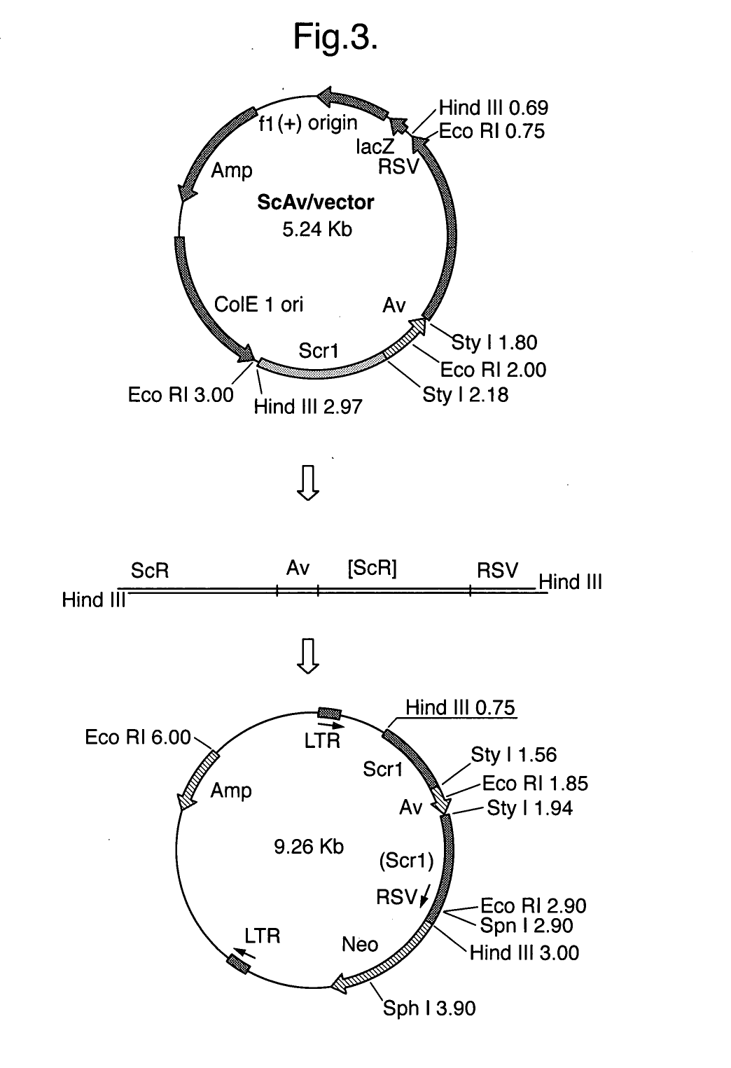

FIGS. 2 and 3 refer to processes used in this Example. More specifically, FIG. 2 shows how the ScR cDNA with an internal RSV promoter was cut from plasmid pLScRNL by HindIII and cloned into a HindIII site of a shuttle vector. FIG. 3 shows how the ScR-avidin-RSV cDNA was cloned into a retrovirus vector pLRNL HindIII site.

The expression of the fusion protein in cells transfected with the vector can be confirmed by Northern blotting and immunocytochemical staining with an antibody raised against avidin.

The experiments revealed that the full mRNA transcript was translated into 55 kDa monomers, which were able to form secondary structures of 110 kDa dimers attached by S—S bonds under non-reducing conditions. Approximately 110 kDa dimeric and 55 kDa monomeric peptides were detected, using denaturing conditions. The result is comparable to the computer calculation for the monomeric fusion protein, 45 kDa. In non-denaturing conditions (i.e., using acetylation prior to Western blotting), the strongest signal was approximately 220 kDa which was denatured to an approximately 110 kDa dimer and a 55 kDa monomer, suggesting the formation of tetramers. The presence of the 220 kDa protein was also verified using chemical cross-linkers, e.g., NHS-esters. The results show that avidin remains soluble and is capable of forming tetramers even when attached to membrane-spanning domains of endocytotic receptors.

The fusion protein was shown to be a functional protein capable of binding FITC-biotin when analyzed by confocal microscopy and atomic force microscopy. Untransduced cells and cells transfected with a retrovirus vector containing the LacZ gene were used as controls. No non-specific binding of biotin probes to LacZ-transduced control cells was detected by atomic force microscopy. As expected, the transfected cells showed specific binding that was repeatably measurable in unfixed samples. The measured binding forces were multiples of the average 149±19pN (mean±sd), which is, as also expected, within the range of the earlier reported biotin-streptavidin binding force of 160 pN (Florin et al. (1994), Science 264:415-417).

Functionality of the construct can also be confirmed in vivo by showing the binding of fluorescently-labeled biotin molecules to cells having the fusion protein construct, using FACS analysis.

The functional activity of the fusion protein in vivo was analyzed in a rat malignant glioma model. BT4C wild-type glioma cells were implanted intracranially in the right corpus callosum at a depth of 2.5 mm in the brain of inbred BDIX female rats. The growth of tumors was monitored frequently with high resolution MRI (magnetic resonance imaging). Three weeks after tumor cell inoculations, pseudotyped retrovirus carrying cDNA for the fusion protein or LacZ gene in titers of 2×10⁶ cfu/ml and 1.3×10⁶ cfu/ml, respectively, was transferred into the tumor, firstly at a depth of 2.5 mm and then at a depth of 1.5 mm, with a 10 minute interval. Gene transfer was repeated after two days of growth. Animals were sacrificed and perfusion-fixed with 4% PFA 3 days after the last injection. Brains were removed and divided at the injection site into two coronal pieces, sectioned on ice and analyzed with immunoreactivity against anti-avidin antibody. The results showed that the fusion protein was expressed in vivo in rat malignant glioma. Protein was detected in glioma cells and in ring-like structures resembling vascular endothelial cells in tumor blood vessels.

### EXAMPLE 2

The procedure of Example 1 was adapted, to provide the construct of SEQ ID NO: 3 encoding the functional fusion protein. The capability of this fusion protein to bind biotinylated ligands was studied in vitro and in vivo. Its functionality has been demonstrated in three different animal models: 1) a nude mouse model bearing subcutaneous tumours, 2) a rat malignant glioma model, and 3) a transgenic mouse model expressing the fusion protein in the endothelium of splenic blood vessels. Three different imaging methods were used, for the detection of biotinylated ligands (immunohistochemistry, SPECT and MRI). In addition, preliminary results demonstrate therapeutic efficacy of the fusion protein in nude mice bearing subcutaneous tumours.

Radiolabeled biotin (Technetium-99m-diethylenetriamine-pentaacetic acid (^{99m}Tc-DTPA) label) was administered systemically to glioma-bearing rats. Rats were imaged by planar gamma camera. Systemic administration of ^{99m}Tc-DTPA was done through tailvein injection. Two hours after injection of the ^{99m}Tc-DTPA, the rats were perfused and imaged.

Transgenic mice expressing the fusion protein on the endothelium of the spleen were investigated in a MRI study. Animals were injected with biotinylated-USPIO into the tailvein of transgenic mice (non-biotinylated-USPIO served as control).

Studies in nude mice bearing subcutaneously growing tumors show that the fusion protein is capable of binding (concentrating) enough biotinylated-DOTA-Yttrium to the tumor region in order to have a therapeutic effect. The tumor volume declines after day 4, whereas the control groups show tumor growth.

In vitro studies using biotinylated paclitaxel-filled nanoparticles were induced, in order to evaluate if nanoparticulate drug carriers could be used in combination the fusion protein. It was demonstrated that the number of viable cells was reduced by over 50% compared to the control cells. In addition, no therapeutic effect was seen in cells which were transduced and treated with a non-biotinylated version of the paclitaxel-filled nanoparticles.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method for enhancing action of a drug on a cell when administered to the cell, wherein the drug is modified by biotinylation and wherein the cell is transduced to express a fusion protein comprising a membrane-spanning domain of an endocytotic receptor and an extracellular domain that comprises biotin-binding activity.

2. The method according to claim 1, wherein the fusion protein further comprises a cytoplasmic domain.

3. The method according to claim 1, wherein the extracellular domain comprises a biotin-binding domain of avidin or streptavidin.

4. The method according to claim 1, wherein the receptor is scavenger receptor class A.

5. The method according to claim 1, wherein the fusion protein comprises SEQ ID NO: 2.

6. A method for treating a disease in a patient, said method comprising administering to said patient a biotinylated molecule useful in the treatment of said disease, wherein said biotinylated molecule is targeted to a target site comprising a fusion protein comprising a membrane-spanning domain of an endocytotic receptor and an extracellular domain that comprises biotin-binding activity, and wherein said biotinylated molecule exerts its effect on said target site.

7. The method according to claim 6, wherein the fusion protein further comprises a cytoplasmic domain.

8. The method according to claim 6, wherein the extracellular domain comprises a biotin-binding domain of avidin or streptavidin.

9. The method according to claim 6, wherein the receptor is scavenger receptor class A.

10. The method according to claim 6, wherein the fusion protein comprises SEQ ID NO: 2.


SEQUENCE LISTING (5 sequences)

SEQ ID NO: 1 - DNA, 5177 bp, artificial sequence
  plasmid encoding fusion protein
tttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagccaagcttgggctgcaggtcgactctagaggatcaattcggcacgagtaaatcggtgctgccgtctttaggacatatgaagtatggcacagtgggatgactttcctgatcagcaagaggacactgacagctgtacagagtctgtgaagttcgatgctcgctcagtgacagctttgcttcctccccatcctaaaaatggcccaactcttcaagagaggatgaagtcttataaaactgcactgatcaccctttatctcattgtgtttgtagttctcgtgcccatcattggcatagtggcagctcagctcctgaaatgggaaacgaagaattgcacggttggctcagttaatgcagatatatctccaagtccggaaggcaaaggaaatggcagtgaagatgaaatgagatttcgagaagctgtgatggaacgcatgagcaacatggaaagcagaatccagtatctttcagataatgaagccaatctcctagatgctaagaatttccaaaatttcagcataacaactgatcaaagatttaatgatgttcttttccagctaaattccttactttcctccatccaggaacatgagaatatcataggggatatctccaagtcattagtaggtctgaacaccacagtacttgatttgcagttcagtattgaaacactgaatggcagagtccaagagaatgcatttaaacaacaagaggagatgcgtaaattagaggagcgtatatacaatgcatcagcagaaattaagtctctagatgaaaaacaagtatatttggaacaggaaataaaaggggaaatgaaactgttgaataatatcactaatgatctgaggctgaaggattgggaacattctcagacattgaaaaatatcactttactccaaggtgccagaaagtgctcgctgactgggaaatggaccaacgatctgggctccaacatgaccatcggggctgtgaacagcagaggtgaattcacaggcacctacatcacagccgtaacagccacatcaaatgagatcaaagagtcaccactgcatgggacacaaaacaccatcaacaagaggacccagcccacctttggcttcaccgtcaattggaagttttcagagtccaccactgtcttcacgggccagtgcttcatagacaggaatgggaaggaggtcctgaagaccatgtggctgctgcggtcaagtgttaatgacattggtgatgactggaaagctaccagggtcggcatcaacatcttcactcgcctgcgcacacagaaggagtgagtgagtgaccaaggtcctcctggactccaggtgaaaaaggagatagaggccctcctggacaaaatggtataccaggctttccaggtctaataggtactccaggtcttaaaggtgatcggggggatctctggtttacctggagttcgaggattcccaggaccaatggggaagaccgggaagccaggacttaatggacaaaaaggccagaagggagaaaaagggagtggaagcatgcaaagacaatctaatacagtccgactggtgggtggcagcggccctcacgaaggcagagtggagatttttcacgaaggccagtggggtacggtgtgtgacgaccgctgggaactgcgtggaggactggtcgtctgcaggagcttgggatacaaaggtgttcaaagtgtgcataagcgagcttattttggaaaaggtacgggtccaatatggctgaatgaagtattttgtttcgggaaagagtcatccattgaagagtgcagaattagacagtggggtgtgagagcctgttcgcacgacgaagatgctgggggtcactttgcacctacataatgcatcatattttcattcacattttttaaactgttataaagtgatttttttcctttgcttcactaaaatcagcttaattaatatttaagaaactaagaattttatccacagaaaaggaatatttaaaaatcactggataaacatataaaatagcttcatatttgcttcaaataccagaaccatttcaacttctctaggtttttaagtggctcgtgccgaattgatcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacagcaaacgccatttgaccattcaccacattggtgtgcacctccaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgg

SEQ ID NO: 2 - protein, 400 aa, artificial sequence
  fusion protein
MAQWDDFPDQQEDTDSCTESVKFDARSVTALLPPHPKNGPTLQERMKSYKTALITLYLIVFVVLVPIIGIVAAQLLKWETKNCTVGSVNADISPSPEGKGNGSEDEMRFREAVMERMSNMESRIQYLSDNEANLLDAKNFQNFSITTDQRFNDVLFQLNSLLSSIQEHENIIGDISKSLVGLNTTVLDLQFSIETLNGRVQENAFKQQEEMRKLEERIYNASAEIKSLDEKQVYLEQEIKGEMKLLNNITNDLRLKDWEHSQTLKNITLLQGARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 3 - DNA, 7164 bp, artificial sequence
  plasmid encoding fusion protein
gaattcataccagatcaccgaaaactgtcctccaaatgtgtccccctcacactcccaaattcgcgggcttctgcctcttagaccactctaccctattccccacactcaccggagccaaagccgcggcccttccgtttctttgcttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagccaagcttkggctgcaggtcgactctagactgacatggcggatccacctgctgcagagatggtgcacgcaacctccccgctgctgctgctgctgctgctcagcctggctctggtggctcccggcctctctgccagaaagtgctcgctgactgggaaatggaccaacgatctgggctccaacatgaccatcggggctgtgaacagcagaggtgaattcacaggcacctacatcacagccgtaacagccacatcaaatgagatcaaagagtcaccactgcatgggacacaaaacaccatcaacaagaggacccagcccacctttggcttcaccgtcaattggaagttttcagagtccaccactgtcttcacgggccagtgcttcatagacaggaatgggaaggaggtcctgaagaccatgtggctgctgcggtcaagtgttaatgacattggtgatgactggaaagctaccagggtcggcatcaacatcttcactcgcctgcgcacacaacggctccaagccaagggccatcgtggtggctcctgttcatggcttcatgtactggactgactggggaactcccgccaagatcaagaaagggggcctgaatggtgtggacatctactcgctggtgactgaaaacattcagtggcccaatggcatcaccctaggtctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggaggattaagaacatcaacagcatcaactttgacaaccccgtctatcagaagaccacagaggatgaggtccacatttgccacaaccaggacggctacagctacccctcgagagaacaaaaactaatctcagaagaagacctctgagtgagtgaggatcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacagcaaacgccatttgaccattcaccacattggtgtgcacctccaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 4 - DNA, 7164 bp, artificial sequence; identical to SEQ ID NO: 3
  plasmid encoding fusion protein

SEQ ID NO: 5 - DNA, 7164 bp, artificial sequence; identical to SEQ ID NO: 3
  plasmid encoding fusion protein